(19) United States
(12) Patent Application Publication
(10) Pub. No.: US 2012/0238502 A1
(43) Pub. Date: Sep. 20, 2012

(54) ANTI-FIBROTIC HYDROGEL COMPOSITIONS

(76) Inventor: Sebastianus Gerardus Kluijtmans, Tilburg (NL)

(21) Appl. No.: 13/513,895

(22) PCT Filed: Nov. 22, 2010
(86) PCT No.: PCT/GB10/51933

§ 371 Date: Jun. 5, 2012

(30) Foreign Application Priority Data
Dec. 8, 2009 (GB) 0921460.2

(51) Int. Cl. A61K 38/39

(52) U.S. Cl. 514/17.2

(57) ABSTRACT

A cross linked recombinant gelatin composition for the controlled release of a cellular adhesion inhibitory agent.

### FIELD OF INVENTION

The present invention relates to cross-linked recombinant gelatin hydrogel compositions comprising cellular adhesive inhibitory agents, to methods of making these compositions and methods of inhibiting cellular adhesion through use of such compositions. These hydrogel compositions are useful for delivery of the cellular adhesive inhibitory agent to a site in need of such inhibition.

### BACKGROUND OF THE INVENTION

When injury or wounds occur in the human body, the body naturally reacts through mechanisms to repair the injury and close the wound. Many of these mechanisms are effective and beneficial. An example of such beneficial repair is epidermal regeneration in response to scratches, minor lacerations, and minor burns to the skin. However, in situations involving major injury, such as surgery, the body's repair mechanism can result in the overgrowth of scar tissue. This can lead to serious complications such as surgical adhesions.

Surgical adhesions frequently occur following abdominal surgery and can generally be described as the binding of scarred tissue to adjacent tissue. The incidence of adhesions following abdominal surgery is cumulative with multiple surgeries. A clinically important example of detrimental scar formation occurs with peridural fibrosis. This condition leads to recurrent low back pain after lumbar laminectomy and discectomy (Cauchoix et al., 1978, Spine 3:256-259; Jackson, 1971, J. Bone Joint Surg. 53B:409-616; Pheasant, 1985, Orthop. Clin. North Am. 6:319-329; Yong-Hing et al., 1980, Spine 5:59-64). In this condition tissue scar formation restricts nerve root mobility and has been correlated with recurrent radicular pain, often in the same location as the previously herniated disk (Benoist, M. et al., 1980, Spine 5:432-436).

Given the problems associated with cellular adhesion, various methods have been suggested to prevent formation of such adhesions for example the application of a fine fabric barrier around the organs near a surgical site prior to completing the surgery.

U.S. Pat. No. 5,605,938 to Roufa et al., which is incorporated herein by reference in its entirety, discloses the use of certain anionic polymers as inhibitors of scar formation, particularly surgical adhesions. Roufa et al. further disclose that these anionic polymers also inhibit invasion of the cells associated with detrimental healing processes (i.e., inhibit fibroblast invasion). Thus regulating the healing process and preventing fibrosis.

Roufa et al. disclose the use of certain adhesive proteins in anchoring the inhibitory anionic polymer at the site where inhibitory or regulatory activity is desired. These adhesive proteins are generally proteins containing a substantial amount of dihydroxyphenylalanine (DOPA) and hydroxyl-containing amino acid residues, such as fibrin-based products or fragments of polyphenol adhesion protein from mussel, barnacle, or oyster.

U.S. patent application US 2007-016622 to Zong et al, discloses the use of a cross-linked matrix comprising cross-linked electrophilic and nucleophilic polyethylene glycol molecules to achieve better control over the delivery of the active component. They disclose the entrapment of the anionic polymer in this matrix resulting in a favourable time release profile for the anionic polymer. The matrix may also comprise another non-cross-linked carrier material and the use of chitosan, collagen or gelatin as a carrier material is disclosed.

However further improvements of these compositions incorporating cellular adhesion inhibitory agents are desirable, particularly compositions that improve the controlled delivery of the active component. The present invention achieves this.

### GENERAL DEFINITIONS

The term “comprising” is to be interpreted as specifying the presence of the stated parts, steps or components, but does not exclude the presence of one or more additional parts, steps or components.

Reference to an element by the indefinite article “a” or “an” does not exclude the possibility that more than one of the element is present, unless the context clearly requires that there be one and only one of the elements. The indefinite article “a” or “an” thus usually means “at least one”.

The terms “protein” or “polypeptide” or “peptide” are used interchangeably and refer to molecules consisting of a chain of amino acids, without reference to a specific mode of action, size, three-dimensional structure or origin.

“Gelatin” as used herein refers to any gelatin, whether extracted by traditional methods or recombinant or biosynthetic in origin, or to any molecule having at least one structural and/or functional characteristic of gelatin. Gelatin is currently obtained by extraction from collagen derived from animal (e.g., bovine, porcine, rodent, chicken, equine, piscine) sources, e.g., bones and tissues. The term encompasses both the composition of more than one polypeptide included in a gelatin product, as well as an individual polypeptide contributing to the gelatin material. Thus, the term recombinant gelatin as used in reference to the present invention encompasses both a recombinant gelatin material comprising gelatin polypeptides, as well as an individual gelatin polypeptide.

Polypeptides from which gelatin can be derived are polypeptides such as collagens, procollagens, and other polypeptides having at least one structural and/or functional characteristic of collagen. Such a polypeptide could include a single collagen chain, or a collagen homotrimer or heterotrimer, or any fragments, derivatives, oligomers, polymers, or subunits thereof, containing at least one collagenous domain (i.e. a domain characterized by a high level of repeat Gly-X-Y regions, wherein X and Y are independently any amino acid). The term specifically contemplates engineered sequences not found in nature, such as altered collagen sequences, e.g. a sequence that is altered, through deletions, additions, substitutions, or other changes, from a naturally occurring collagen sequence. Such sequences may be obtained from suitable altered collagen polynucleotide constructs, etc.

A “cross-linking agent” as described herein refers to a composition comprising a cross-linker. “Cross-linker” as used herein refers to a reactive chemical compound that is able to introduce covalent intra- and inter- molecular bridges in organic molecules.

A “hydrogel” refers to a network of polymer chains comprising a substantial amount of water. Depending on the application, i.e. the desired release profile of the active agent and the mechanical stress to which the hydrogel is to be subjected, several types of hydrogels can be used. For example hydrogels that are very stiff and inelastic contain 40-60% of water, hydrogels that are elastic but still rigid contain 60-85% of water and hydrogels that are soft and very elastic containing 85-99% of water.

The terms “tissue adhesion”, “scar formation”, “fibrosis” and “fibroblast invasion” refer to biological healing processes which are considered detrimental to the medically beneficial aim of certain surgical procedures and need to be controlled accordingly.

### DETAILED DESCRIPTION OF THE INVENTION

The present invention is described more fully hereinafter. However this invention may be embodied in many different forms and should not be construed as being limited to the embodiments as set forth herein.

The present invention provides a composition comprising:
(a) at least one cellular adhesion inhibitory agent; and
(b) a cross-linked recombinant gelatin hydrogel matrix wherein the recombinant gelatin comprises at least 0.3 mmol/g lysine and/or hydroxylysine residues before cross-linking and at least 0.15 mmol/g free amine after cross-linking.

The compositions of the present invention generally comprise a cross-linked hydrogel matrix and at least one cellular adhesion inhibitory agent associated, either chemically or physically, with the cross-linked hydrogel matrix. The cellular adhesion inhibitory agent can be any agent effective for inhibiting adhesion of a biological material to another biological material or a non-biological material. Preferably, the cellular adhesion inhibitory agent is an agent effective for inhibiting fibrosis.

Particularly useful as cellular adhesion inhibitory agents in the present invention are those biocompatible anionic polymers known to be effective for inhibiting scar formation, in particular surgical adhesion, and also known to be effective for inhibiting fibrosis in general. Such polymers are useful to inhibit fibroblast invasion, thus regulating the healing process and preventing fibrosis. The polymers are also useful for inhibiting glial cell invasion, bone growth, and neurite outgrowth.

Multiple biocompatible anionic polymers are known in the art, and any of these polymers may be used in the compositions of the present invention. Preferably the cellular adhesion inhibitory agent(s) is/are selected from the group consisting of alginate; chondroitan sulfate, dermatan sulfate, dextran sulfate, hyaluronic acid, heparin, heparin sulfate, keratan sulfate, and pentosan polysulfate. The above-noted anionic polymers may be used either alone or together, in any combination. Accordingly, in the compositions of the present invention, the cellular adhesion inhibitory agent can include one of the above anionic polymers. Alternatively, the cellular adhesion inhibitory agent can include two or more of the above anionic polymers. Further, the cellular adhesion inhibitory agent can include one or more of the above anionic polymers in combination with one or more additional agents known to be useful for inhibiting cellular adhesion. It is preferred that the cellular adhesion inhibitory agent is dextran sulfate, pentosan polysulfate or a mixture thereof.

The inhibitory agent can further include disaccharides of one or more of the anionic polymers. Further, the inhibitory agent can include glycosaminoglycans and proteoglycans including one or more of the anionic polymers.

Anionic polymers for use in the present invention can be obtained from natural sources (e.g., proteoglycans), and can be used as found in nature or purified. Additionally, the anionic polymer can be prepared synthetically, such as through chemical derivatization. For example, the polyglucose polymer dextran can be treated by boiling in sulfuric acid and esterifying with chlorosulfonic acid to produce dextran sulfate. Biocompatible anionic polymers are readily available from commercial sources.

The cellular adhesion inhibitory agent should be present in the compositions of the present invention in an amount sufficient to at least partially inhibit cellular adhesion.

In addition to a cellular adhesion inhibitory agent, as described above, the compositions of the present invention further comprise a cross-linked recombinant gelatin hydrogel matrix. The hydrogel matrix is particularly useful for facilitating a favorable release profile for the cellular adhesion inhibitory agent. As such, the hydrogel matrix can be formulated for delivery of the cellular adhesion inhibitory agent to a site wherein cellular adhesion inhibition is required so that such delivery can be delayed, or prolonged, as required for the specific use. Generally hydrogels for controlled release show a two step release profile. A first phase of quick diffusional release short after introduction in vivo. In a second phase there is a gradual sustained release caused by the gradual biodegradation of the hydrogel in the body.

For inhibition of cellular adhesion it is desirable to reduce the diffusional release of the first phase and to have a more gradual release over a longer period in the second phase. The hydrogel matrix of the invention is formulated such that the release of the cellular adhesion inhibition agent is slower, but maintained over a longer period of time, with such time period being adjustable.

The inventors of the current invention have surpisingly found that a cross-linked recombinant gelatin hydrogel wherein the recombinant gelatin is high in lysine and/or hydroxylysine residues before cross-linking and which retains at least 0.15 mmol/g free amine groups after cross-linking is beneficial for achieving the above mentioned desired release profile. Preferably in the recombinant gelatin hydrogel matrix the recombinant gelatin comprises at least 0.3 mmol/g lysine residues before cross linking.

This in contrast to the teachings in the prior-art which is silent on the particular benefits of using cross-linked hydrogels comprising gelatin peptides that retain a defined amount of free amine. Moreover the published US patent application 2007-016622 to Zong et al, teaches the use of a cross-linked matrix comprising cross-linked electrophilic and nucleophilic polyethylene glycol molecules to achieve better control over the delivery of the active component. However the use of biomaterials is desirable over synthetic polyethylene glycol since there is evidence that polyethylene glycol activates the complement pathway which can lead to anaphylaxis in sensitive subjects. (Hamad I et al., Mol Immunol. 2008 Dec; 46(2):225-32). Furthermore the composition of Zong et al, requires cross-linking of the hydrogel in the presence of the cellular adhesion inhibitory agent to entrap this agent within the matrix of the hydrogel to achieve control over the delivery of the active component. The hydrogel of the current invention can be prepared separately before combining it with an adhesion inhibitory agent.

Using animal-tissue prepared gelatins to produce cross-linked hydrogel compositions has several problems. A major disadvantage is that upon administration the release of the cellular adhesion inhibitory agent is far from linear and does not exhibit the required prolonged release. This less than optimal release profile is generally attributed to the inhomogeneous nature of the natural derived polymers, i.e. their very broad molecular weight distribution and potential presence of animal derived impurities. The latter is becoming of growing importance with respect to safety for the use in humans. The present invention is directed to circumvent these disadvantages of the prior art.

The use of recombinant gelatins is of medical benefit in comparison to the conventionally produced gelatins from animal sources. Safety issues, such as concern over potential immunogenic, e.g. antigenic and allergenic responses, have arisen. The inability to completely characterize, purify, or reproduce the animal-derived gelatin mixtures used currently is of ongoing concern in the pharmaceutical and medical communities. Additional safety concerns exist with respect to bacterial contamination and endotoxin loads resulting from the extraction and purification processes. Recombinantly produced gelatins are a solution to these problems. Moreover the recombinant technology allows the design of gelatin-like proteins with superior characteristics for example, but not limited to, low immunogenicity, improved cell attachment, optimal iso-electric point and controlled biodegradability. The mono-disperse nature or in other words uniformity in structure and size of the recombinant gelatins means that the mesh-size of the cross-linked hydrogel and the consequent release-rate of the cellular adhesion inhibitory agent will be uniform, which is greatly desired. EP 0926543, EP 1014176 and WO 01/34646, and also specifically the examples of EP 0926543 and EP 1014176, describe recombinant gelatins and their production methods, using methylotrophic yeasts, in particular Picha pastoris.

Another important advantage of recombinant gelatins is that the amino acid sequence can be manipulated to create certain characteristics. Examples of characteristics that can now be manipulated are (i) the amount of cross-linkable amino acids (for example the amount of (hydroxy)lysines), (ii) the glycosylation pattern (for example the absence of threonine and/or serine amino acids in certain triplets results in the absence of glycosylation), (iii) the size of the recombinant gelatin, (iv) the charge density of the recombinant gelatin can be amended (for example charged amino acids, such as asparagine (Asn), aspartic acid (Asp), glutamine (Gln), glutamic acid (Glu) or lysine (Lys) can be introduced or left out) and as such the release profile of a fibrosis inhibiting agent can be influenced or (v) the biodegradability can be amended by the presence or absence of cleavage sites for metalloproteases.

As mentioned above one important characteristic of a recombinant gelatin is the amount of cross-linkable amino acids, such as the amount of (hydroxy)lysine groups and the amount of carboxylic acid groups derived from aspartic and glutamic acid.

In a preferred embodiment, the invention provides a composition wherein in the recombinant gelatin hydrogel matrix the recombinant gelatin comprises at least 0.40 mmol/g lysine and/or hydroxylysine (preferably lysine) residues before cross-linking, more preferably at least 0.60 mmol/g and especially at least 0.80 mmol/g.

Clearly if more cross-linkable groups are available, the amount of cross-links can in principle be higher if compared to a situation in which less cross-linkable groups are present. The lower limit of cross-linkable groups is that amount that can still result in the formation of a gel. The amount of cross-linkable groups in principle also determines the mesh size which is a measure of the average “pore size” of the entangled/cross-linked gelatin network at physiological conditions (pH 7.4, 37° C. and 300 mOsm/L). Finally, the amount of cross-linked groups determines the biodegradability of the formed controlled release composition. By using a recombinant gelatin, the amount of cross-linkable groups can be influenced and thus the gel mesh size and biodegradability can be manipulated.

The 0.15 mmol/g free amine groups present in the hydrogel of the present invention after cross-linking may comprise lysine and hydroxylysine residues which did not react during cross-linking. The inventors of the current invention have found that the retained free amine residues in the cross-linked hydrogel matrix give rise to better controllable release profiles of the anionic polymers used as cellular adhesion inhibitory agent. Without being bound to theory, these better controllable release profiles can be explained by the electrostatic interaction between anionic polymers used as fibrosis inhibiting agent and the positively charged free amines of the cross-linked gelatin hydrogel slowing the diffusional release, resulting in a more sustained and gradual release. Other methods to increase free amines in gelatin hydrogels such as chemically modifications or adding other polymers e.g. polylysine may influence the release profile in similar fashion but give rise to unwanted side-effects on the biocompatibility and lack of immungenicity of the hydrogel of the current invention.

The amount of free amines in the hydrogel may be determined by any method know in the art. Preferably the amount of free amines in the hydrogel determined using the TNBS (2,4,6-trinitrobenzenesulfonicacid) method (see for example Gilbert and Kim, J. Biomed. Matter. Res. 24, 1221, 1990).

In a further embodiment of the present invention the recombinant gelatin is a gelatin enriched in RGD motifs. The term ‘RGD-enriched gelatins’ in the context of this invention means that the gelatinous polypeptides have a certain level of RGD motifs, calculated as a percentage of the total number of amino acids per molecule and a more even distribution of RGD. RGD-enriched gelatins in the context of this invention are described in WO 04/085473 and WO 08/103041 which are incorporated herein by reference. Preferably the recombinant gelatin comprises at least one RGD motif and more preferably at least three and especially at least five.

In another further embodiment the recombinant gelatins used in the composition of the present invention are recombinant gelatins with an iso-electric point above 5, preferably an iso-electric point above 6 and more preferably an iso-electric point above 7. By adjusting the iso-electric point of gelatin that is cross-linked to form the hydrogel an attraction or repulsion of cellular adhesion inhibitory agent can be achieved, especially in case of anionic polymers.

In another further embodiment the recombant gelatines used in the compositions of the present invention have a molecular weight of at least 25 kDa, more preferably of at least 35 kDa and most preferably of at least 50 kDa.

An important feature of the cross-linked hydrogel of the present invention is, that the polymer used in the hydrogel formation should be biodegradable and so not require invasive surgical methods for its removal after the release of the fibrosis inhibiting agent. Moreover biodegradability is another important factor in the gradual release of the cellular adhesive inhibitory agents (especially fibrosis inhibiting agents) used in the composition. A priori it is not obvious whether recombinant gelatins will be broken down by the same mechanisms causing degradation of natural gelatins. It is known that natural gelatins and collagens are degraded in the human body by proteases and more specifically matrix-metalloproteinases (MMP). Matrix metalloproteinases (MMP's) are zinc-dependent endopeptidases. The MMP's belong to a larger family of proteases known as the metzincin superfamily. Collectively they are capable of degrading all kinds of extracellular matrix proteins, but also can process a number of bioactive molecules. An important group of MMP's are the collagenases. These MMP's are capable of degrading triple-helical fibrillar collagens into distinctive ¾ and ¼ fragments. These collagens are the major components of bone and cartilage, and MMP's are the only known mammalian enzymes capable of degrading them. Traditionally, the collagenases are: MMP-1 (interstitial collagenase), MMP-8 (neutrophil collagenase), MMP-13 (collagenase 3) and MMP-18 (collagenase 4). Another important group of MMP's is formed by the gelatinases. The main substrates of these MMP's are type IV collagen and gelatin, and these enzymes are distinguished by the presence of an additional domain inserted into the catalytic domain. This gelatin-binding region is positioned immediately before the zinc binding motif, and forms a separate folding unit which does not disrupt the structure of the catalytic domain. The two members of this sub-group are: MMP-2 (72 kDa gelatinase, gelatinase-A) and MMP-9 (92 kDa gelatinase, gelatinase-B). However, International patent application WO/2008103045 discloses that a recombinant gelatin that does not comprise a known cleavage site for MMP was enzymatically degradable by human matrix metalloproteinase 1 (MMP1). Apparently many more types of recombinant gelatin than predicted can be degraded. Therefore the cross-linked hydrogel compirising recombinant gelatin exhibits the required gradual biodegradation desirable in a composition for the prevention of cellular adhesion.

The preparation of the hydrogel of the compostion of the current invention, can be performed by any method known in the art. Suitable cross-linking agents are those known in the art such as chemical cross-linkers selected from aldehyde compounds such as formaldehyde and glutaraldehyde, carbodiimide, di-aldehyde di-isocyanate, ketone compounds such as diacetyl and chloropentanedion, bis (2-chloroethylurea), 2-hydroxy-4,6-dichloro-1,3,5-triazine, reactive halogen-containing compounds disclosed in U.S. Pat. No. 3,288,775, carbamoyl pyridinium compounds in which the pyridine ring carries a sulfate or an alkyl sulfate group disclosed in U.S. Pat. Nos. 4,063,952 and 5,529,892, divinylsulfones, and the like and S-triazine derivatives such as 2-hydroxy-4,6-dichloro-s-triazine. In a preferred embodiment the method for preparing the hydrogel of the composition of the current invention is performed by cross-linking with a water soluble carbodimide. In an even more preferred embodiment the recombinant gelatin is cross-linked using 1-ethyl-3-[3-dimethylaminopropyl]carbodiimide hydrochloride (EDC or EDAC). Cross-linking reaction conditions vary depending on which cross-linking agent is used and would be well known to a skilled person.

It is preferred that compositions according to the present invention are suitable for use in the prevention of cellular adhesion. It is particularly preferred that compositions according to the present invention are suitable for use in the prevention of fibrosis.

A preferred composition the according to the present invention comprises;
(a) 0.1 to 8 percent by weight of at least one cellular adhesion inhibitory agent; and
(b) 92 to 99.9 percent by weight of a cross-linked recombinant gelatin hydrogel matrix wherein the recombinant gelatin comprises at least 0.3 mmol/g lysine residues before cross-linking and at least 0.15 mmol/g free amine after cross-linking.

Compositions according to the present invention may be prepared by any method which would be known to the skilled person. Preferably compositions according to the present invention are prepared by a method which comprises:
(a) mixing the recombinant gelatin and the cellular adhesion inhibitory agent(s) to obtain a mixture; and
(b) cross-linking the mixture.

Compositions of the present invention may also preferably be prepared by a method which comprises:
(a) cross-linking the recombinant gelatin to give a hydrogel matrix; and
(b) contacting the hydrogel matrix with the cellular adhesion inhibitory agent(s). The invention is illustrated in the following, non-limiting examples wherein which all parts and percentages are by weight unless otherwise stated.

### EXAMPLES

An example of the invention is illustrated in the accompanying figure.

FIG. 1 shows the rate of release of dextran sulfate from various cross-linked recombinant gelatins.

Curve 1 shows the release profile with the hydrogel of Example 1a.

Curve 2 shows the release profile with the hydrogel of Example 1c.

Curve 3 shows the release profile with the hydrogel of Example 1b.

### Example 1

### Preparation of Cross-Linked Recombinant Gelatin Using Various Cross-Linkers

The recombinant gelatin used in these examples is CBE3 and was disclosed in WO2008103041. The molecular weight of this recombinant gelatin is about 51200 Daltons.

### Example 1a

### Use of Hexamethvlene Diisocyanate (HMDIC) as a Cross-Linking Agent

A sponge of CBE3 was prepared by freeze drying a 1% aqueous solution of CBE 3 at pH 10. The sponge thus obtained was cross-linked by immersion in ethanol containing 0.0075% HMDIC for 24 hours at ambient temperatures. Residual cross-linker was removed by washing three times in excess pure ethanol. After drying the sponge was mashed into small pieces by a shredder (Retsch ZM1) to yield a dry powder (particle size <400 μm).

### Example 1 b

Use of 1-ethyl-3-[3-dimethylaminopropyl]carbodiimide hydrochloride (EDC) as a Cross Linking Agent

A 10% aqueous solution of CBE 3 was adjusted to pH 4.7 by the addition of hydrochloric acid and cross-linked by adding an aqueous solution of 50% EDC in a ratio of 1 g EDC solution/g gelatin. The cross-linking reaction proceeded for 24 hours at ambient temperatures. The resulting gel was diluted with water and mashed using an Ultra-Turrax™ for 10 minutes (IKA T10, S10N8G probe). The gel was then precipitated in pure ethanol, decanted, and washed three times with pure ethanol. The material was dried in a vacuum oven at 40° C. to yield a dry powder.

### Example 1 c

### Use of Formaldehyde as a Cross Linking Agent

A 10% aqueous solution of CBE3 was adjusted to pH 10 using 1 M sodium hydroxide and subsequently crosslinked by the addition of a formaldehyde (12.3 M) solution in methanol (0.14 ml g/g gelatin). The resultant gel was precipitated in pure ethanol, decanted, and washed three times with pure ethanol. The material was dried in a vacuum oven at 40° C. to yield a dry powder.

### Example 2

### Gel Preparation Process

Cross-linked recombinant (1g) gelatin powder, prepared as described in Examples 1 a, 1 b and 1 c, was dispersed in 9 ml isotonic phosphate buffer at pH 7.4 containing 2% dextrane sulfate (average MW 40.000). The resulting dispersions were autoclaved for 15 minutes at 121° C. to obtain three hydrogels.

Release of Dextrane Sulfate from a Gelatin Hydrogels

The release of dextrane sulfate from the gelatin hydrogel was measured according to methods described in WO2007/016622. Hydrogel (1g) was dispersed in 50m1 of saline phosphate buffer. The flask was stoppered and gently swirled at 37° C. At time points 0, 3, 7, and 24 hours aliquots were taken and filtered through a 0.2 μm filter. Blanks were obtained by using gels with the same cross-linked gelatins that did not contain any dextrane sulfate. The dextrane sulfate concentration in the filtered aliquots was quantified by ICP-OES based on sulfur determination. Data were corrected by subtraction of the blank values.

### RESULTS

Release patterns of gels of differently cross-linked gelatins are shown in FIG. 1. It is apparent from FIG. 1 that the dextrane sulfate-release strongly depends on the type of agent used to cross-link the gelatin. For example, EDC cross-linked recombinant gelatin gels release less than 10% of dextrane sulfate by diffusion. Here it is likely that the dextrane sulfate release in vivo will be mainly by degradation (hydrolytic and enzymatic) of the hydrogel. In contrast for the HMDIC cross-linked hydrogel the diffusional release is completed up to 70% after 24 hours. Interestingly it seems that the remainder 30% of the dextran sulfate is not released by diffusion and hence will be released in vivo by degradation. The release of dextran sulfate from formaldehyde cross-linked gels is in-between HMDIC and EDC cross-linked gels.

The level of amines before and after cross-linking were determined using the TNBS method (2,4,6-trinitrobenzene sulfonic acid, see e.g. Gilbert and Kim, J. Biomed. Matter. Res. 24, 1221, 1990). Resulting values for some characteristic gels are shown in Table 1.

TABLE 1

| | Non X- | | | |
|---|---|---|---|---|
| | linked CBE3 | HMDIC | FA | EDC |
| Free amine after cross | 0.64 | 0.48 | 0.46 | 0.56 |
| linking | | | | |
| (mmol/1 g dry gelatin) | | | | |
| Total amount of | — | 6 | 10 | 18 |
| dextran sulfate | | mg/g gel | mg/g gel | mg/g gel |
| trapped in the hydrogel | | | | |

In conclusion it is shown that by using a cross-linked recombinant gelatin hydrogel, with a tailored content of free amine groups, after cross-linking, the desired type of release pattern of a cellular adhesion inhibitory agent can be obtained.

1-29. (canceled)

30. A composition comprising:
(a) at least one cellular adhesion inhibitory agent wherein the cellular adhesion inhibitory agent(s) is/are selected from the group consisting of alginate, chondroitan sulfate, dermatan sulfate, dextran sulfate, hyaluronic acid, heparin, heparin sulfate, keratin sulfate, and pentose polysulfate; and
(b) a cross-linked recombinant gelatin hydrogel matrix wherein the recombinant gelatin comprises at least 0.3 mmol/g lysine and/or hydroxylysine residues before cross-linking and at least 0.15 mmol/g free amine after cross-linking.

31. A composition according to claim 30 wherein the cellular adhesion inhibitory agent is dextran sulfate, pentosan polysulfate or a mixture thereof.

32. A composition according to claim 30 wherein in the recombinant gelatin hydrogel matrix the recombinant gelatin comprises at least 0.40 mmol/g lysine and/or hydroxylysine residues before cross-linking.

33. A composition according to claim 30 wherein in the recombinant gelatin hydrogel matrix the recombinant gelatin comprises at least 0.80 mmol/g lysine and/or hydroxylysine residues before cross-linking.

34. A composition according to claim 30 wherein in the recombinant gelatin hydrogel matrix the recombinant gelatin comprises at least 0.3 mmol/g lysine residues before cross-linking.

35. A composition according to claim 30 wherein the recombinant gelatin comprises at least one RGD motif.

36. A composition according to claim 30 wherein the recombinant gelatin has an iso-electric point above 7.

37. A composition according to claim 30 wherein the recombinant gelatin has a molecular weight of at least 50 kDa.

38. A composition according to claim 30 wherein the recombinant gelatin is cross-linked using 1-ethyl-3-[3-dimethylaminopropyl]carbodiimide hydrochloride.

39. A composition according to claim 30 comprising
(a) 0.1 to 8 percent by weight of at least one cellular adhesion inhibitory agent wherein the cellular adhesion inhibitory agent(s) is/are selected from the group consisting of alginate, chondroitan sulfate, dermatan sulfate, dextran sulfate, hyaluronic acid, heparin, heparin sulfate, keratin sulfate, and pentose polysulfate; and
(b) 92 to 99.9 percent by weight of a cross-linked recombinant gelatin hydrogel matrix wherein the recombinant gelatin comprises at least 0.3 mmol/g lysine residues before cross-linking and at least 0.15 mmol/g free amine after cross-linking.

40. A composition according to claim 30 for use in the prevention of cellular adhesion.

41. A composition according to claim 30 for use in the prevention of fibrosis.

42. A method for preparing the compositions as described in claim 30 which comprises:
(a) mixing the recombinant gelatin and the cellular adhesion inhibitory agent(s) wherein the cellular adhesion inhibitory agent(s) is/are selected from the group consisting of alginate, chondroitan sulfate, dermatan sulfate, dextran sulfate, hyaluronic acid, heparin, heparin sulfate, keratin sulfate, and pentose polysulfate to obtain a mixture; and
(b) cross-linking the mixture.

43. A method for preparing the compositions as described in claim 30 which comprises:
(a) cross-linking the recombinant gelatin to give a hydrogel matrix; and
(b) contacting the hydrogel matrix with the cellular adhesion inhibitory agent(s) wherein the cellular adhesion inhibitory agent(s) is/are selected from the group consisting of alginate, chondroitan sulfate, dermatan sulfate, dextran sulfate, hyaluronic acid, heparin, heparin sulfate, keratin sulfate, and pentose polysulfate.
